(19) United States
(12) Patent Application Publication
(10) Pub. No.: US 2025/0272462 A1
(43) Pub. Date: Aug. 28, 2025

(54) DETERMINATION DEVICE AND CALCULATION METHOD

(71) Applicants: Preferred Networks, Inc., Tokyo (JP); ENEOS Corporation, Tokyo (JP)

(72) Inventors: So TAKAMOTO, Tokyo (JP); Wenwen LI, Tokyo (JP)

(73) Assignees: Preferred Networks, Inc., Tokyo (JP); ENEOS Corporation, Tokyo (JP)

(21) Appl. No.: 19/191,342

(22) Filed: Apr. 28, 2025

Related U.S. Application Data
(63) Continuation of application No. PCT/JP2023/038951, filed on Oct. 27, 2023.

(30) Foreign Application Priority Data
Oct. 28, 2022 (JP) 2022-173543

(51) Int. Cl. G06F 30/28; G06F 30/27

(52) U.S. Cl. CPC G06F 30/28; G06F 30/27

(57) ABSTRACT

A determination device in one example of the present disclosure includes at least one memory and at least one processor. The processor inputs at least one first atomic structure to a trained model and generates at least one of a first energy or a first force corresponding to the first atomic structure. The processor calculates at least one of a second energy or a second force corresponding to the first atomic structure based on the first atomic structure, a given parameter set, and a model of a potential. The processor determines a parameter set by updating the given parameter set based on at least one of a difference between the first energy and the second energy or a difference between the first force and the second force.

### CROSS-REFERENCE TO RELATED APPLICATIONS

This application is based upon and claims the benefit of priority from Japanese Patent Application No. 2022-173543, filed on Oct. 28, 2022, and International Patent Application No. PCT/JP2023/038951 filed on Oct. 27, 2023; the entire contents of all of which are incorporated herein by reference.

### FIELD

The present disclosure relates to a determination device and a calculation method.

### BACKGROUND

There is a simulation using a classical molecular dynamics potential (hereinafter referred to as “classical potential”). For example, a simulation using an optimized potentials for liquid simulations (OPLS) potential is executed by applying OPLS-all atom (AA) parameters to a formula representing a desired three-dimensional atomic structure OPLS potential (See, for example, William L. Jorgensen, David S Maxwell, Julian Tirado-Rives: “Development and Testing of the OPLS All-Atom Force Field on Conformational Energetics and Properties of Organic Liquids”, J. Am. Chem. Soc. 1996, 118, 11225-11236).

A parameter set (for example, OPLS-AA parameters) in the classical potential is calculated in advance by, for example, quantum chemistry calculation such as density functional theory (DFT).

However, in the simulation using the classical potential (for example, the OPLS potential), there has often been a problem that molecules of interest which are fast but available are limited. In addition, there has been a problem that it takes a lot of time to determine a parameter set in a classical potential.

### SUMMARY

A determination device according to an embodiment includes at least one memory and at least one processor. The at least one processor is configured to input a first atomic structure into a function having a parameter set and generate at least one of a second energy or a second force corresponding to the first atomic structure, and update the parameter set based on at least one of a difference between the second energy and a first energy generated by inputting the first atomic structure into a trained model, or a difference between the second energy force and a first force generated by inputting the first atomic structure into the trained model.

### BRIEF DESCRIPTION OF THE DRAWINGS

FIG. 1 is a block diagram illustrating an example of a hardware configuration of a determination device according to an embodiment;

FIG. 2 is a diagram illustrating examples of functional blocks in a processor according to the embodiment;

FIG. 3 is a flowchart illustrating an example of a procedure of an optimum parameter determination process according to the embodiment;

FIG. 4 is a flowchart illustrating an example of a procedure of a generality validation process according to a first application example of the embodiment; and

FIG. 5 is a flowchart illustrating an example of a procedure of an optimum parameter determination process added to the processing procedure illustrated in FIG. 4 according to a second application example of the embodiment.

### DETAILED DESCRIPTION

Hereinafter, an embodiment will be described in detail with reference to the drawings.

### Embodiment

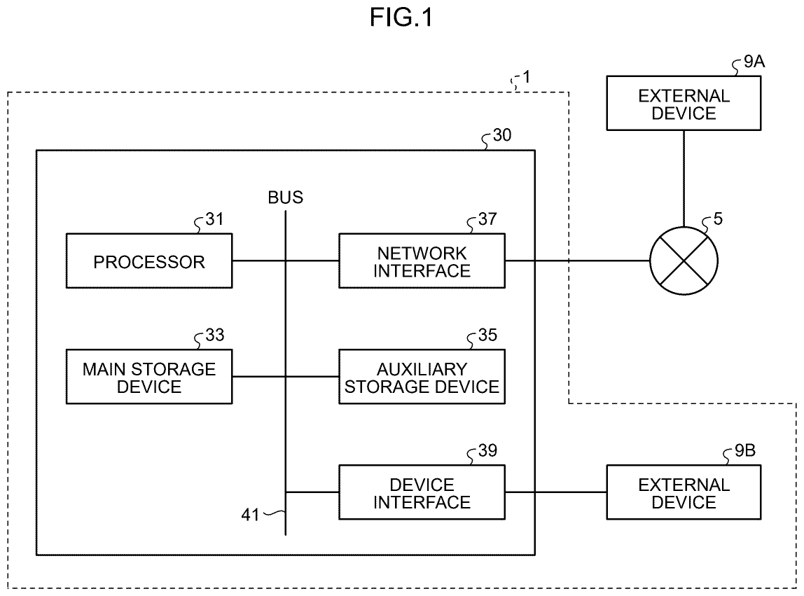

FIG. 1 is a block diagram illustrating an example of a hardware configuration of a determination device 1 according to the embodiment. As illustrated in FIG. 1, the determination device 1 may be connected to an external device 9A via a communication network 5. The determination device 1 may include an external device 9B connected via a device interface 39. The determination device 1 may determine a parameter set in a classical potential related to a three-dimensional atomic structure corresponding to a notation that is input by a user and is indicative of a structure of a substance consisting of a plurality of atoms. The substance is, for example, a molecule. The substance is not limited to a molecule and may be any of various crystals or the like. The notation is, for example, a simplified molecular input line entry system (SMILES) notation input by the user in regard to the substance. The SMILES notation represents, for example, information about a specific molecule (information about atoms and how the atoms are connected) in accordance with given rules. For example, in the case of methane, the SMILES notation represents information about the particle size such that four hydrogen (H) atoms are connected to one carbon (C) atom. The notation is not limited to the SMILES notation and may be another known notation as long as the substance can be uniquely identified. Hereinafter, to make concrete description, it is assumed that information input by the user via an input device to be described below is information (hereinafter referred to as SMILES information) corresponding to the SMILES notation.

The classical potential is defined by a model of a potential expressed by a simple formula and a set of parameters in the formula expressing the model. The classical potential is a function that returns at least one of an energy and a force (that is, an energy and/or a force) in response to input of a structure of an atom. In a simulation using the classical potential, a force acting on an atom can also be output. Since a force is defined as a derivative of a position of the atom with respect to the energy, additional implementation in the simulation is required. However, the force can also be calculated by calculating the energy. There are several types of models of the classical potential, for example, an assisted model building with energy refinement (AMBER) potential (force field) and an optimized potentials for liquid simulations (OPLS) potential (force field). Thus, the model of the classical potential is expressed by a formula corresponding to an AMBER potential (force field) or an OPLS potential. A model that expresses the classical potential is not limited to the AMBER potential or the OPLS potential, and may be another known potential (force field).

The determination device 1 includes a computer 30 and an external device 9B connected to the computer 30 via a device interface 39. In one example, the computer 30 includes a processor 31, a main storage device (memory) 33, an auxiliary storage device (memory) 35, a network interface 37, and a device interface 39. The determination device 1 may be implemented by a computer 30 in which the processor 31, the main storage device 33, the auxiliary storage device 35, the network interface 37, and the device interface 39 are connected via a bus 41.

The computer 30 illustrated in FIG. 1 includes one constituent for each element, but may include the same constituents for one element. Although one computer 30 is illustrated in FIG. 1, software may be installed in a plurality of computers, and each of the computers may execute the same or different processes of the software. In this case, there may be a form of distributed computing in which each computer communicates via the network interface 37 or the like to execute a process. Thus, the determination device 1 according to the embodiment may be configured as a system that implements various functions to be described below by one or more computers executing commands stored in one or more storage devices. The information transmitted from a terminal may be processed by one or more computers provided on a cloud, and a processing result may be transmitted to a terminal such as a display device (display unit) corresponding to the external device 9B.

Various operations of the determination device 1 according to the embodiment may be executed as parallel processes by using one or more processors or by using a plurality of computers via a network. In addition, various operations may be distributed to a plurality of arithmetic cores in the processor and executed as parallel processes. Some of or all the processes, means, and the like according to the present disclosure may be executed by at least one of a processor and a storage device provided on a cloud that can communicate with the computer 30 via a network. As described above, various types to be described below according to the embodiment may be in the form of parallel computing by one or more computers.

The processor 31 may be an electronic circuit (a processing circuit, a processing circuit, a processing circuitry, a central processing unit (CPU), a graphics processing unit (GPU), a field programmable gate array (FPGA), an application specific integrated circuit (ASIC), or the like) including a control device and an arithmetic device of the computer 30. The processor 31 may be a semiconductor device or the like including a dedicated processing circuit. The processor 31 is not limited to an electronic circuit with an electronic logic element, and may be implemented by an optical circuit with an optical logic element. The processor 31 may have an arithmetic function based on quantum computing.

The processor 31 can execute an arithmetic process based on data and software (computer program) input from each device or the like of the internal configuration of the computer 30 and output an arithmetic result and a control signal to each device or the like. The processor 31 may control each constituent included in the computer 30 by executing an operating system (OS), an application, or the like of the computer 30.

The determination device 1 according to the embodiment may be implemented by one or more processors 31. Here, the processor 31 may be one or more electronic circuits disposed on one chip or may be one or more electronic circuits disposed on two or more chips or two or more devices. When a plurality of electronic circuits is used, the electronic circuits may communicate in a wired or wireless manner.

The main storage device 33 is a storage device that stores instructions executed by the processor 31, various types of data, and the like, and information stored in the main storage device 33 is read by the processor 31. The auxiliary storage device 35 is a storage device other than the main storage device 33. Note that these storage devices mean arbitrary electronic constituents capable of storing electronic information, and may be semiconductor memories. The semiconductor memory may be either a volatile memory or a nonvolatile memory. The storage device that stores various types of data used for the determination device 1 according to the embodiment may be implemented by the main storage device 33 or the auxiliary storage device 35 or may be implemented by an internal memory built into the processor 31. For example, the storage unit according to the embodiment may be implemented by the main storage device 33 or the auxiliary storage device 35.

A plurality of processors may be connected (coupled) or a single processor 31 may be connected to one storage device (memory). A plurality of storage devices (memories) may be connected (coupled) to one processor. When the determination device 1 according to the embodiment includes at least one storage device (memory) and a plurality of processors connected (coupled) to the at least one storage device (memory), at least one processor among the plurality of processors may have a configuration in which the at least one processor is connected (coupled) to the at least one storage device (memory). This configuration may be implemented by a storage device (memory) included in a plurality of computers and the processor 31. Further, a storage device (memory) may be integrated with the processor 31 (for example, a cache memory including an L1 cache and an L2 cache).

The network interface 37 is an interface for connection to the communication network 5 in a wireless or wired manner. As the network interface 37, an appropriate interface such as an interface conforming to an existing communication standard may be used. The network interface 37 may exchange information with the external device 9A connected via the communication network 5. The communication network 5 may be any of a wide area network (WAN), a local area network (LAN), a personal area network (PAN), and the like, or a combination thereof, as long as information is exchanged between the computer 30 and the external device 9A. Examples of the WAN include the Internet, examples of the LAN include IEEE 802.11 and Ethernet (registered trademark), and examples of the PAN include Bluetooth (registered trademark) and near field communication (NFC).

The device interface 39 is an interface such as a universal serial bus (USB) directly connected to an output device such as a display device, an input device, and the external device 9B. The output device may include a speaker that outputs sound or the like.

The external device 9A is a device connected to the computer 30 via a network. The external device 9B is a device directly connected to the computer 30.

In one example, the external device 9A or the external device 9B may be an input device (input unit). The input device is, for example, a device such as a camera, a microphone, a motion capture, any of various sensors, a keyboard, a mouse, or a touch panel, and provides the acquired information to the computer 30. The external device 9A or 9B may be a device or the like such as a personal computer, a tablet terminal, or a smartphone that includes an input unit, a memory, and a processor.

In one example, the external device 9A or 9B may be an output device (output unit). The output device may be, for example, a display device (display unit) such as a liquid crystal display (LCD), a cathode ray tube (CRT), a plasma display panel (PDP), or an organic electro luminescence (EL) panel or may be a speaker that outputs sound or the like. The external device 9A or 9B may be a device or the like such as a personal computer, a tablet terminal, or a smartphone that includes an output device, a memory, and a processor.

The external device 9A or 9B may be a storage device (memory). For example, the external device 9A may be a network storage or the like, and the external device 9B may be a storage such as an HDD.

The external device 9A or 9B may be a device that has some functions of the constituents of the determination device 1 according to the embodiment. Thus, the computer 30 may transmit or receive some of or all processing results of the external device 9A or 9B.

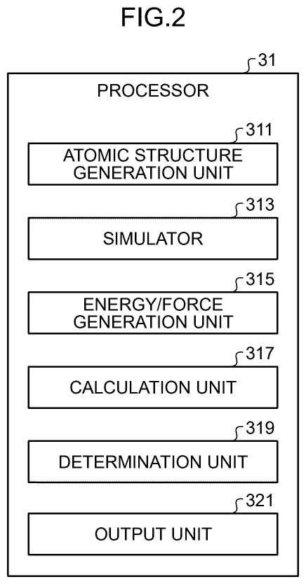

FIG. 2 is a diagram illustrating examples of functional blocks in the processor 31. The processor 31 includes, for example, an atomic structure generation unit 311, a simulator 313, an energy/force generation unit 315, a calculation unit 317, a determination unit 319, and an output unit 321 for functions that are implemented by the processor 31. The functions implemented by the atomic structure generation unit 311, the simulator 313, the energy/force generation unit 315, the calculation unit 317, the determination unit 319, and the output unit 321 are stored as computer programs in, for example, the main storage device 33 or the auxiliary storage device 35. The processor 31 can implement functions related to the atomic structure generation unit 311, the simulator 313, the energy/force generation unit 315, the calculation unit 317, the determination unit 319, and the output unit 321 by reading and executing programs stored in the main storage device 33, the auxiliary storage device 35, or the like.

The atomic structure generation unit 311 may generate a three-dimensional initial atomic structure (hereinafter referred to as an initial structure) based on information about SMILES (hereinafter referred to as SMILES information) input by an input device. The initial structure corresponds to an arrangement of atoms that is obtained by three-dimensionally arranging plural atoms related to a substance indicated by SMILES notation. The initial structure may be referred to as a second atomic structure. A known technique can be appropriately used for a process of generating the initial structure based on the SMILES information, and thus description thereof will be omitted.

The simulator 313 may be implemented by simulation software that executes calculation of molecular dynamics. Hereinafter, to make concrete description, it is assumed that the simulator 313 is implemented by a large-scale atomic/molecular massively parallel simulator (LAMMPS). Means for implementing the simulator 313 is not limited to the LAMMPS and may be implemented by another molecular dynamics simulator.

The simulator 313 may generate one or more three-dimensional atomic structures (for example, the first atomic structures) used for the energy/force generation unit 315 and the calculation unit 317 to be described below based on the initial structure and the given parameter set in accordance with the SMILES information. Thus, the simulator 313 generates one or more structures of a specific molecule by, for example, a molecular dynamics simulation. The present disclosure is not limited to the above dynamics calculation as long as a large number of structures are generated by the classical potential. For example, as another example, one or more structures of a specific molecule may be generated by the Monte Carlo simulation as a classical potential. In addition, another method may be used for generating one or more structures of specific molecules. For example, the simulator 313 can generate a plurality of three-dimensional atomic structures although slightly deformed since atoms vibrate when the temperature is finite in a single molecule.

Accordingly, the simulator 313 may generate one or more three-dimensional atomic structures based on the initial structure and a given parameter set related to the classical potential corresponding to the initial structure. The given parameter set may be a set of one or more parameters in the classical potential corresponding to the SMILES information. The one or more parameters are, for example, numerical values corresponding to a bond, an angle, a force constant, an equilibrium coupling length, an equilibrium coupling shell, an electric charge, and the like, and also correspond to, for example, a list of several hundred to tens of thousands of the numerical values. When the classical potential is an OPLS potential, the given parameter set is an OPLA-AA parameter. The OPLS-AA parameter is generated by a known technique, and thus description thereof will be omitted. The given parameter set may be a set of random values.

Specifically, the simulator 313 may read, from the main storage device 33 or the auxiliary storage device 35, a given parameter set in accordance with a formula that expresses a model of a potential indicating the OPLS potential corresponding to the initial structure based on the model of the potential in the classical potential and the SMILES information. The simulator 313 may generate one or more three-dimensional atomic structures by using the read given parameter set and the initial structure. One or more three-dimensional atomic structures may be referred to as a trajectory.

The given parameter set is not limited to the OPLS-AA parameter. For example, when the classical potential is an AMBER potential, the given parameter set corresponds to a set of parameters included in a formula that expresses the classical potential in the AMBER potential according to the initial structure.

The energy/force generation unit 315 may be implemented by, for example, a trained model that inputs each of three-dimensional atomic structures or one three-dimensional atomic structure output from the simulator 313 and outputs a first energy and/or a first force corresponding to each of the three-dimensional atomic structures or one three-dimensional atomic structure. The trained model may be implemented by, for example, a trained neural network potential (hereinafter referred to as NNP for the neural network potential). The trained NNP is, for example, a general-purpose neural network potential that can correspond to any atomic structure. The trained NNP is, for example, a trained graph neural network that includes an input layer, one or more graph convolution (graph convolution) layers, and an output layer.

Each of three-dimensional atomic structures or one three-dimensional atomic structure is input as input information to the input layer. The input information is, for example, a positional relationship (such as coordinates) between atoms in a substance, a structural relationship (for example, a structural formula of a compound consisting of atoms, and the like) of the atoms, an atomic number of each of the atoms, all charges by the atoms, or the like. The input layer may generate a graph indicating a relationship between the atoms based on the input information. The graph may include a node (also referred to as a vertex) for each of atoms and a side (also referred to as a link) indicating a structural relationship among the atoms and connecting nodes. The graph is expressed as, for example, a matrix. The input layer may determine feature amounts allocated to nodes corresponding to the atoms based on the input information and a correspondence table that converts the atoms into vectors. Accordingly, the input layer may input a structure of a substance consisting of atoms which are represented by feature amounts, to the first-stage graph convolution layer as a graph.

The one or more graph convolution layers may maintain an input graph to repeat the graph convolution of the feature amounts within a preset cutoff range (also referred to as a cutoff radius). For one or more graph convolution layers, a pre-convolution range for the graph may be preset as a cutoff radius. The cut-off radius may be able to be appropriately set in accordance with a user's instruction via the input device. The graph convolution layer at the last stage may output a vector indicating a feature amount corresponding to a plurality of atoms.

The output layer may output a first energy and/or a first force (that is, at least one of the first energy and the first force) corresponding to each of the three-dimensional atomic structures or one three-dimensional atomic structure based on the feature amount calculated by the graph convolution layer at the last stage. The details of processing content of the NNP will not be described because an existing technique can be used as appropriate.

Accuracy of the first energy and/or the first force output from the NNP conforms to, for example, accuracy of a calculation result by quantum chemical calculation such as density functional theory (hereinafter referred to as the DFT) used for training the NNP. Even when data output from the trained model is only the first energy, the energy/force generation unit 315 can output the first force by a derivative of a position with respect to the first energy.

The calculation unit 317 may calculate the second energy and/or the second force corresponding to each of the three-dimensional atomic structures or one three-dimensional atomic structure based on each of the three-dimensional atomic structures or one three-dimensional atomic structure generated by the simulator 313, a given parameter set, and a model of the classical potential. The calculation unit 317 is implemented by, for example, a differentiable library such as pytorch. Specifically, the calculation unit 317 may calculate the second energy and/or the second force for each of three-dimensional atomic structures or one three-dimensional atomic structure by substituting a given parameter set into the model of the classical potential.

The calculation of the second energy and/or the second force by the calculation unit 317 is not limited to a differentiable library such as a pytorch. For example, any software may be used as long as the software is a library that can calculate the second energy and/or the second force in each of the three-dimensional atomic structures or one three-dimensional atomic structure by using the model of the classical potential into which the given parameter set is substituted. Hereinafter, to make concrete description, software used for the calculation unit 317 is assumed to be a differentiable library. The software is software (hereinafter referred to as energy/force software) that calculates the second energy and/or the second force corresponding to each of the three-dimensional atomic structures or one three-dimensional atomic structure by using each of the three-dimensional atomic structures or one three-dimensional atomic structure, a given parameter set, and a model of the classical potential. The energy/force software may be implemented as application software.

In a case where the calculation unit 317 is implemented by a neural network as one example of a differentiable library, a set of weights for the neural network corresponds to a given parameter set. At this time, the neural network corresponds to a classical potential, and the set of weights for the neural network corresponds to the given parameter set. At this time, the data input to the neural network is each of three-dimensional atomic structures or one three-dimensional atomic structure generated by the simulator 313, and the data output from the neural network is the second energy and/or the second force.

The determination unit 319 may adjust the given parameter set related to the classical potential such that a difference between the first energy and the second energy (hereinafter referred to as a first difference) and/or a difference between the first force and the second force (hereinafter referred to as a second difference) is reduced in each of the three-dimensional atomic structures or one three-dimensional atomic structure generated by the simulator 313 to determine a parameter set related to the classical potential. The first and second differences correspond to errors when the first energy and the first force are used as training data (also referred to as correct answer data).

Thus, the determination unit 319 may adjust the given parameter set so as to minimize an error and then determine a parameter set corresponding to the classical potential in the initial structure (hereinafter referred to as an optimum parameter set). In other words, the determination unit 319 may determine the optimum parameter set by executing an optimization process of minimizing an error on the given parameter. An objective function in the optimization process corresponds to the error.

For example, in a case where the calculation unit 317 is implemented by a differentiable library, the determination unit 319 may calculate a derivative of each of the parameters included in the given parameter set with respect to an error to reduce the first difference and/or the second difference, namely, an error, and adjust the given parameter set. Specifically, the determination unit 319 may execute adjustment of the given parameter set on each of three-dimensional atomic structures to generate an adjusted parameter set (hereinafter referred to as an adjustment parameter set). Accordingly, the determination unit 319 may generate a plurality of adjustment parameter sets corresponding to the plurality of three-dimensional atomic structures. Subsequently, the determination unit 319 may determine an optimum parameter set through an approximation process such as a least squares method on the plurality of adjustment parameter sets.

In a case where calculation unit 317 is implemented by a non-differentiable library, the determination unit 319 may randomly change a given parameter and thereby determine a parameter set that has a minimum error as the optimum parameter set. The determination of the optimum parameter set by the determination unit 319 corresponds to, for example, training of the OPLS parameter set using an error and the OPLA-AA parameter set as an initial value. In other words, the optimum parameter set corresponds to a trained parameter set that is trained from the given parameter set by using the error.

The output unit 321 may convert the optimum parameter set into a specific file format in accordance with a simulation in which the classical potential is used. The output unit 321 may output the optimum parameter set to the main storage device 33 and/or the auxiliary storage device 35 in the converted file format. The output unit 321 may output the optimum parameter set in the converted file format to the external device 9A via the communication network 5. At this time, the external device 9A corresponds to, for example, a simulator that executes simulation by using an optimization parameter and a classical potential.

For example, in a case where the simulation is LAMMPS and the classical potential is a LAMMPS potential, the output unit 321 converts the optimum parameter set into a LAMMPS data file. The output unit 321 may output the LAMMPS data file to the external device 9A that is capable of executing LAMMPS.

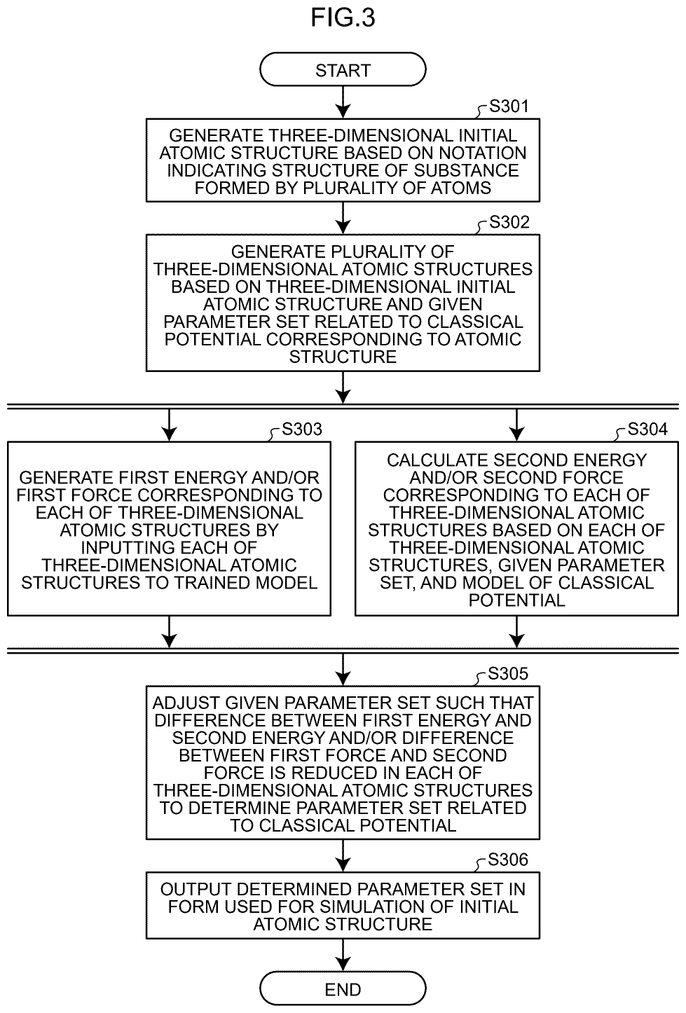

The configuration of the determination device 1 has been described above. Hereinafter, a process of determining the optimum parameter set by the determination device 1 (hereinafter referred to as an optimum parameter determination process) will be described with reference to FIG. 3.

FIG. 3 is a flowchart illustrating an example of a procedure of an optimum parameter determination process.

### Optimum Parameter Determination Process

### (Step S301)

SMILES information about a substance desired by the user may be input to the determination device 1 through an input device. At this time, the model of the classical potential may be selected in response to an instruction by the user through the input device. The information about the substance is not limited to the SMILES information and may be information in accordance with another notation regarding the substance. At this time, the processor 31 may read the given parameter set corresponding to the SMILES information from the main storage device 33, the auxiliary storage device 35, or the like. In addition, the processor 31 may read a model of the selected classical potential (for example, an OPLS potential or an AMBER potential) or a preset model of the classical potential from the main storage device 33, the auxiliary storage device 35, or the like.

The atomic structure generation unit 311 may generate a three-dimensional initial atomic structure based on a notation indicating a structure of a substance consisting of a plurality of atoms. Thus, the atomic structure generation unit 311 may generate the initial structure based on the SMILES information. The atomic structure generation unit 311 outputs the generated initial structure, the given parameter set, and the model of the classical potential to the simulator 313. In addition, the atomic structure generation unit 311 may output the given parameter set and the model of the classical potential to the calculation unit 317.

### (Step S302)

The simulator 313 may generate one or more three-dimensional atomic structures based on the three-dimensional initial atomic structure and the given parameter set related to a classical potential corresponding to the atomic structure. Thus, the simulator 313 may generate one or more three-dimensional atomic structures by a molecular dynamics simulation using the initial structure and the given parameter set. The simulator 313 may output one or more three-dimensional atomic structures to the energy/force generation unit 315 and the calculation unit 317.

### (Step S303)

The energy/force generation unit 315 may input each of the three-dimensional atomic structures or one three-dimensional atomic structure to the trained model to generate the first energy and/or the first force corresponding to each of the three-dimensional atomic structures or one three-dimensional atomic structure. Specifically, the energy/force generation unit 315 may generate the first energy and/or the first force as an output from the NNP by inputting each of the three-dimensional atomic structures or one three-dimensional atomic structure to the NNP. Accordingly, the energy/force generation unit 315 may generate one or more first energies and/or one or more first forces corresponding to the three-dimensional atomic structures or one three-dimensional atomic structure. The energy/force generation unit 315 may output the one or more first energies and/or the one or more first forces to the determination unit 319.

### (Step S304)

The calculation unit 317 may calculate the second energy and/or the second force corresponding to each of the three-dimensional atomic structures or one three-dimensional atomic structure based on each of the three-dimensional atomic structures or one three-dimensional atomic structure, the given parameter set, and the model of the classical potential. Specifically, the calculation unit 317 may read the energy/force software from the main storage device 33, the auxiliary storage device 35, or the like. Subsequently, the calculation unit 317 may calculate the second energy and/or the second force corresponding to each of the three-dimensional atomic structures or one three-dimensional atomic structure by applying each of the three-dimensional atomic structures or one three-dimensional atomic structure, the given parameter set, and the model of the classical potential to the energy/force software and executing the energy/force software.

Accordingly, the calculation unit 317 may generate one or more second energies and/or one or more second forces corresponding to one or more three-dimensional atomic structures. The calculation unit 317 may output one or more second energies and/or one or more second forces to the determination unit 319.

### (Step S305)

The determination unit 319 may adjust the given parameter set such that a difference between the first energy and the second energy and/or a difference between the first force and the second force is reduced in one or each of the three-dimensional atomic structures to determine a parameter set (optimum parameter set) related to the classical potential. Thus, the determination unit 319 determines the optimum parameter set by executing an optimization process of minimizing a plurality of errors corresponding to one or more three-dimensional atomic structures.

Specifically, the given parameters may be adjusted using a differentiation operation in the energy/force software or the like to minimize an error in each of the three-dimensional atomic structures or one three-dimensional atomic structure. Accordingly, the determination unit 319 may generate a plurality of adjustment parameter sets by adjusting a given parameter for one or more three-dimensional atomic structures. Subsequently, the determination unit 319 may determine the optimum parameter set by executing an optimization process on a value of each of items of the parameters in the adjustment parameter sets.

### (Step S306)

The output unit 321 may output the determined parameter set in a form used for a simulation of the initial atomic structure. Thus, the output unit 321 may convert the optimum parameter set into a specific file format in accordance with the simulation in which the classical potential corresponding to the initial structure or the SMILES information is used. The output unit 321 may output the optimum parameter set expressed in the converted specific file format to the main storage device 33, the auxiliary storage device 35, and/or the external device 9A. The optimum parameter determination process in this step is completed, and then the user may execute a simulation in which the optimum parameter set and the classical potential expressed in the specific file format is used.

The determination device 1 according to the embodiment may generate one or more three-dimensional atomic structures based on the initial structure and the given parameter set related to the classical potential corresponding to the initial structure, input each of the three-dimensional atomic structures or one three-dimensional atomic structure to a trained model (for example, an NNP) to generate the first energy and/or the first force corresponding to each of the three-dimensional atomic structures or one three-dimensional atomic structure, calculate the second energy and/or the second force corresponding to each of the three-dimensional atomic structures or one three-dimensional atomic structure based on each of the three-dimensional atomic structures or one three-dimensional atomic structure, the given parameter set, and the model of the classical potential, and adjust (optimize) the given parameter set such that the difference between the first energy and the second energy (first difference) and/or the difference between the first force and the second force (second difference) is reduced in each of the three-dimensional atomic structures or one three-dimensional atomic structure to determine the parameter set related to the classical potential.

Accordingly, in the determination device 1 according to the embodiment, it is possible to generate an optimum parameter set with higher accuracy than an existing given parameter set according to the atomic structure of the substance desired by the user and the model of the classical potential corresponding to the atomic structure. In the determination device 1 according to the embodiment, the first energy and/or the first force corresponding to correct data in the training of the given parameter set can be generated by using a trained general-purpose NNP that is faster than a DFT and has accuracy conforming to the DFT. Therefore, it is possible to generate the optimum parameter set in a short time with high accuracy. Accordingly, in the determination device 1 according to the embodiment, it is possible to effectively use the trained NNPs in the generation of the classical potential.

From the above, by the determination device 1 according to the embodiment, it is possible to determine the parameter set in any classical potential used for a simulation in a short time and with high accuracy. For example, in the determination device 1, it is possible to determine the parameter set in any classical potential used for a simulation on a spot in a short time and with high accuracy in accordance with the classical potential, a substance, or the like desired by the user. Therefore, in the simulator using the optimum parameters determined by the determination device 1, it is possible to execute a high-speed simulation using the classical potential with high accuracy for any of various substances (molecules and the like).

### First Application Example

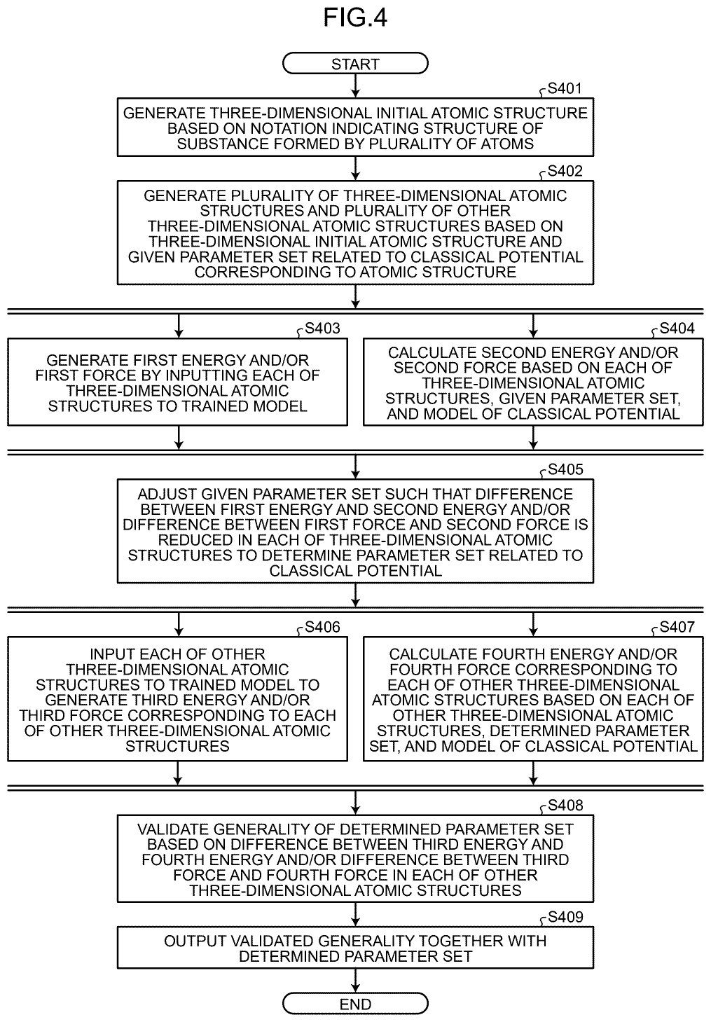

In the present application example, generality (namely, generalization performance) of the optimum parameter determined by the determination unit 319 is validated. Hereinafter, description will be made on the assumption that validation of generality is executed by the determination unit 319. The validation of generality may be executed by another unit (for example, a validation unit) different from the determination unit 319. In such a case, the validation unit is installed in the processor 31 as one function of the processor 31. Hereinafter, a process of validating generality of the optimum parameter in the present application example (hereinafter referred to as a generality validation process) will be described with reference to FIG. 4.

FIG. 4 is a flowchart illustrating an example of a procedure of a generality validation process. Steps S401, S403, S404, and S405 in FIG. 4 are similar to steps S301, S303, S304, and S305 in FIG. 3, respectively, and thus description thereof will be omitted.

### Generality Validation Process

### (Step S402)

The simulator 313 may generate one or more three-dimensional atomic structures and one or more three-dimensional atomic structures (hereinafter referred to as other atomic structures) different from one or more three-dimensional atomic structures based on the three-dimensional initial atomic structure and the given parameter set related to the classical potential corresponding to the atomic structure. The other atomic structures correspond to the third atomic structure. Thus, the simulator 313 may generate one or more three-dimensional atomic structures and one or more other atomic structures by a molecular dynamics simulation using the initial structure and the given parameter set. The simulator 313 may output one or more three-dimensional atomic structures and one or more other atomic structures to the energy/force generation unit 315 and the calculation unit 317. One or more other atomic structures may be used for validating generality of the optimum parameter set.

### (Step S406)

The energy/force generation unit 315 may input each of the other atomic structures or a single other atomic structure to the trained model to generate a third energy and/or a third force corresponding to each of the other atomic structures or the single other atomic structure. Specifically, the energy/force generation unit 315 may input each of the other atomic structures or the single other atomic structure to the NNP to generate the third energy and/or the third force as an output from the NNP. Accordingly, the energy/force generation unit 315 may generate one or more of the third energies and/or the third forces corresponding to one or more of the other atomic structures. The energy/force generation unit 315 may output one or more third energies and/or the third forces to the determination unit 319.

### (Step S407)

The calculation unit 317 may calculate a fourth energy and/or a fourth force corresponding to each of the other atomic structures or a single other atomic structure based on each of the other atomic structures or the single other atomic structure, the parameter set (optimum parameter set) determined in step S405, and the model of the classical potential. Specifically, the calculation unit 317 may apply each of the other atomic structures or the single other atomic structure, the optimum parameter set, and the model of the classical potential to the read energy/force software and execute the energy/force software to calculate the fourth energy and/or the fourth force corresponding to each of the other atomic structures or the single other atomic structure. Accordingly, the calculation unit 317 may generate one or more fourth energies and/or one or more of the fourth forces corresponding to one or more of the other atomic structures. The calculation unit 317 may output the fourth energies and/or the fourth forces to the determination unit 319.

### (Step S408)

The determination unit 319 may validate generality of the determined parameter set based on a difference between the third energy and the fourth energy (hereinafter referred to as a third difference) and/or a difference between the third force and the fourth force (hereinafter referred to as a fourth difference) in each of the other atomic structures or the single other atomic structure. A known technique can be applied to the validation of the generality, and thus the description thereof will be omitted.

### (Step S409)

The output unit 321 may output the validated generality together with the determined parameter set (optimum parameter set) to the main storage device 33, the auxiliary storage device 35, and/or the external device 9A. The output unit 321 may display the validated generality on a display together with the optimum data set expressed in a specific file format.

The determination device 1 according to the first application example of the embodiment may generate one or more of the other atomic structures based on the initial structure and the given parameter set, input each of the other atomic structures or the single other atomic structure to the trained model to generate the third energy and/or the third force corresponding to each of the other atomic structures or the single other atomic structure, calculate the fourth energy and/or the fourth force corresponding to each of the other atomic structures or the single other atomic structure based on the optimum parameter set, each of the other atomic structures or the single other atomic structure, and the model of the classical potential, and validate the generality of the optimum parameter set based on the third difference and/or the fourth difference in each of the other atomic structures or the single other atomic structure. Subsequently, the determination device 1 may output a validation result of generality to the display or the like.

Accordingly, the user can confirm the generality of the optimum parameter set, and can ascertain reliability of the optimum parameter set. Other effects in the present application example are the same as those in the embodiment, and thus description thereof will be omitted.

### Second Application Example

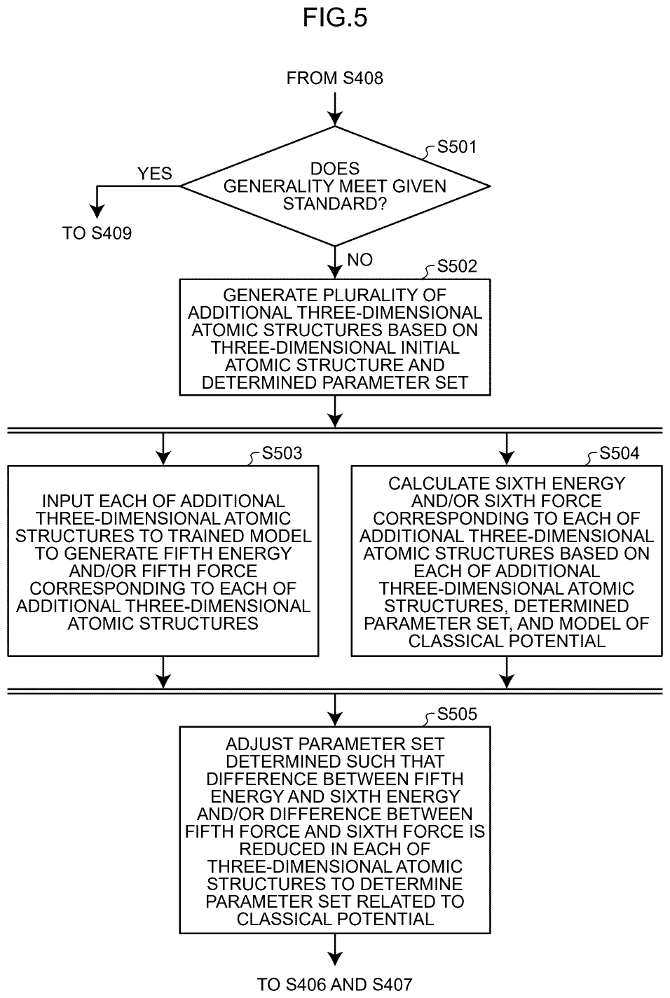

In the present application example, when the generality validated in the first application example does not meet a given standard, the optimum parameters are determined again based on a different three-dimensional atomic structure (for example, the fourth atomic structure). Hereinafter, an optimum parameter determination process in the present application example will be described with reference to FIG. 5. FIG. 5 is a flowchart illustrating an example of a procedure of an optimum parameter determination process added to the processing procedure illustrated in FIG. 4.

### Optimum Parameter Determination Process

### (Step S501)

This step may be executed after step S408 illustrated in FIG. 4. When the generality meets the given standard (Yes in step S501), the process of step S409 is executed. When the generality does not meet the given standard (No in step S501), the process of step S502 may be executed. The determination in this step is executed by, for example, the determination unit 319. The given standard is defined by, for example, thresholds corresponding to the third difference and the fourth difference. The generality then corresponds to the third and fourth differences. Determination of the generality including quantification of the generality is not limited to the above, and a known technique can be applied.

### (Step S502)

The simulator 313 may generate one or more additional three-dimensional atomic structures (hereinafter referred to as an additional atomic structure) based on the three-dimensional initial atomic structure and the determined parameter set. The additional atomic structure corresponds to the fourth atomic structure. Thus, the simulator 313 may generate one or more of the additional atomic structures by a molecular dynamics simulation in which the initial structure and the determined parameter set are used. The simulator 313 may output one or more of the additional atomic structures to the energy/force generation unit 315 and the calculation unit 317. The one or more of the additional atomic structures may be used for further learning the parameter set determined by the determination unit 319.

### (Step S503)

The energy/force generation unit 315 may input each of the additional atomic structures or one additional atomic structure to the trained model to generate a fifth energy and/or a fifth force corresponding to each of the additional atomic structures or one additional atomic structure. Accordingly, the energy/force generation unit 315 may generate one or more of the fifth energies and/or one or more of the fifth forces corresponding to one or more additional atomic structures. The energy/force generation unit 315 may output the fifth energies and/or the fifth forces to the determination unit 319.

### (Step S504)

The calculation unit 317 may calculate a sixth energy and/or a sixth force corresponding to each of the additional atomic structures or one additional atomic structure based on each of the additional atomic structures or one additional atomic structure, the parameter set determined in step S405, and the model of the classical potential. Accordingly, the calculation unit 317 may generate one or more sixth energies and/or one or more sixth forces corresponding to one or more of the additional atomic structures. The calculation unit 317 may output one or more of the sixth energies and/or one or more of the sixth forces to the determination unit 319. The determined parameter set used for this step corresponds to a latest parameter set.

### (Step S505)

The determination unit 319 may adjust the given parameter set such that a difference (hereinafter referred to as a fifth difference) between the fifth energy and the sixth energy and/or a difference (hereinafter referred to as a sixth difference) between the fifth force and the sixth force is reduced in each of the additional atomic structures or one additional atomic structure to determine the parameter set related to the classical potential. Thus, the determination unit 319 may determine the parameter set by executing an optimization process of minimizing one or more errors corresponding to one or more of the additional atomic structures. In this step, the determination unit 319 may update the optimum parameter set. In the process after step S505, the processes of steps S406 and S407 may be repeated.

The determination device 1 according to the second application example of the embodiment may generate one or more of the additional structures based on the initial structure and the determined parameter set when the generality does not meet the given standard, input each of the additional structures or one additional atomic structure to the NPP to generate the fifth energy and/or the fifth force corresponding to each of the additional structures or one additional atomic structure, calculate the sixth energy and/or the sixth force corresponding to each of the additional structures or one additional atomic structure based on the determined parameter set, each of the additional structures or one additional atomic structure, and the model of the classical potential, and adjust the determined parameter set to reduce the fifth difference and/or the sixth difference in each of the additional structures or one additional atomic structure to determine the parameter set (optimum parameter set) related to the classical potential. Subsequently, the determination device 1 may output a validation result of generality to the display or the like.

Accordingly, in the determination device 1, by updating the parameter set until the generality of the determined parameter set meets the given standard, it is possible to generate the parameter set that has the generality that meets the given standard while validating the generality. Thus, as illustrated in FIGS. 4 and 5, in the present application example, the parameter set in the classical potential can be repeatedly trained. According to a modification of the present application example, for example, the optimum parameter set can be determined by using, as the given parameter set, a parameter set to which a numerical value such as 0 or 1 is mechanically assigned.

As described above, in the determination device 1 according to the present application example, even when a substance has no known parameter set such as an OPLS-AA parameter set as the given parameter set, the optimum parameter set can be determined. Other effects in the present application example are similar to those in the embodiment and the first application example, and thus the description thereof will be omitted.

### Third Application Example

In the present application example, an additional structure may be generated over a preset number of times and the determined parameter may be updated without executing determination based on generality in the second application example. Thus, the processing content in step S501 illustrated in FIG. 5 may be determination processing of determining whether or not the optimum parameter has been determined a given number of times. In the present application example, the processes from step S406 to step S409 may be unnecessary. In steps S502 to S505, an additional atomic structure corresponds to a single other atomic structure (for example, the fifth atomic structure), the fifth energy and the fifth force correspond to a seventh energy and a seventh force, respectively, and the sixth energy and the sixth force correspond to an eighth energy and an eighth force, respectively. Thus, detailed description of the present application example is omitted.

For example, the determination device 1 according to the third application example of the embodiment may generate one or more other atomic structures (for example, the fifth atomic structures) different from one or more three-dimensional atomic structures (for example, the first atomic structures) based on the initial structure (for example, the second atomic structure) and the determined parameter set, input each of the other atomic structures or a single other atomic structure to the trained model to generate the seventh energy and/or the seventh force corresponding to each of the other atomic structures or the single other atomic structure, calculate the eighth energy and/or the eighth force corresponding to each of the other atomic structures or the single other atomic structure based on the determined parameter set, the plurality of other atomic structures or the single other atomic structure, and the model of the classical potential, and adjust the determined parameter set such that a difference between the seventh energy and the eighth energy and/or a difference between the seventh force and the eighth force is reduced in each of the other atomic structures or the single other atomic structure to determine the parameter set (optimum parameter set) related to the classical potential. The effects of the present application example are similar to those of the second application example except for the validation of the generality, and thus description thereof will be omitted.

When the technical idea in the embodiment is implemented by a determination method, the determination method includes: generating one or more of the three-dimensional atomic structures based on the three-dimensional initial atomic structure and the given parameter set related to the classical potential corresponding to the atomic structure; inputting each of the three-dimensional atomic structures or one three-dimensional atomic structure to the trained model that inputs each of the three-dimensional atomic structures or one three-dimensional atomic structure and outputs a first energy and/or a first force corresponding to each of the three-dimensional atomic structures or one three-dimensional atomic structure to generate the first energy and/or the first force corresponding to each of the three-dimensional atomic structures or one three-dimensional atomic structure; calculating the second energy and/or the second force corresponding to each of the three-dimensional atomic structures or one three-dimensional atomic structure based on one three-dimensional atomic structure or each of the three-dimensional atomic structures, the given parameter set, and the model of the classical potential; and adjusting the given parameter set such that the difference between the first energy and the second energy and/or the difference between the first force and the second force is reduced in each of the three-dimensional atomic structures or one three-dimensional atomic structure to determine the parameter set related to the classical potential. The procedure and effects of the optimum parameter determination process related to the determination method are similar to those described in the embodiment, and thus the description thereof will be omitted.

When the technical idea in the embodiment is implemented by a determination program, the determination program causes a computer to: generate one or more of the three-dimensional atomic structures based on the three-dimensional initial atomic structure and the given parameter set related to the classical potential corresponding to the atomic structure; input each of the three-dimensional atomic structures or one three-dimensional atomic structure to the trained model that inputs each of the three-dimensional atomic structures or one three-dimensional atomic structure and outputs first energy and/or first force corresponding to each of the three-dimensional atomic structures or one three-dimensional atomic structure to generate the first energy and/or the first force corresponding to each of the three-dimensional atomic structures or one three-dimensional atomic structure; calculate the second energy and/or the second force corresponding to each of the three-dimensional atomic structures or one three-dimensional atomic structure based on each of the three-dimensional atomic structures or one three-dimensional atomic structure, the given parameter set, and the model of the classical potential; and adjust the given parameter set such that the difference between the first energy and the second energy and/or the difference between the first force and the second force is reduced in each of the three-dimensional atomic structures or one three-dimensional atomic structure to determine the parameter set related to the classical potential.

For example, the optimum parameter determination process can also be implemented by installing the determination program in a computer in any of various analysis devices, analysis servers, or the like that analyze an energy and/or a force of an atomic structure consisting of a plurality of atoms and loading the determination program on a memory. At this time, a program that can cause the computer to execute the technique can be distributed by being stored in a storage medium such as a magnetic disk (hard disk or the like), an optical disk (a CD-ROM, a DVD, or the like), or a semiconductor memory. The procedure and effects of the optimum parameter determination process by the determination program are similar to those of the embodiment, and thus the description thereof will be omitted.

Some of or all the apparatuses in the above-described embodiment may be configured by hardware or may be configured through information processing of software (program) executed by a CPU, a GPU, or the like. When an apparatus is configured through information processing of software, the information processing of software may be executed by storing software that realizes at least some functions of each apparatus in the above-described embodiment in a non-transitory storage medium (non-transitory computer readable medium) such as a flexible disk, a compact disc-read only memory (CD-ROM), or a USB memory and causing the computer 30 to read the software. The software may be downloaded via the communication network 5. Further, information processing may be executed by hardware by implementing software in a circuit such as an ASIC or an FPGA.

A type of storage medium storing the software is not limited. The storage medium may be a fixed storage medium such as a hard disk or a memory without being limited to a removable storage medium such as a magnetic disk or an optical disc. The storage medium may be provided inside a computer or may be provided outside a computer.

In the present specification (including the claims), the expression “at least one (one side) of a, b, and c” or “at least one (one side) of a, b, or c” (including similar expressions) includes any of a, b, c, a-b, a-c, b-c, or a-b-c. A plurality of instances may be included for any element, such as a-a, a-b-b, a-a-a-b-b-c-c, or the like. The instances also include addition of other elements other than the listed elements (a, b, and c), such as having d as a-b-c-d.

In the present specification (including claims), a case where an expression such as “data used as an input/based on data/according to data/in response to data” (including similar expressions) is used includes a case unless otherwise specified, a case where various types of data are used as an input and a case where data obtained by executing a certain process on various types of data (for example, noise addition, normalization, intermediate representation of various types of data, and the like) is used as an input. When it is described that any result is obtained “based on data/according to data/in response to data”, the case may include a case where the result is obtained based on only the data and may also include a case where the result is obtained under an influence of other data other than the data, a factor, a condition, a state, and/or the like. The case also includes a case where “outputting data” is described, a case where unless otherwise specified, a case where various types of data are used as outputs, and a case where data obtained by executing a certain process on various types of data (for example, noise addition, normalization, intermediate representation of various types of data, and the like) is output.

In the specification (including the claims), when the terms “connected” and “coupled” are intended, the terms are intended as open-ended terms including any of direct connection/coupling, indirect connection/coupling, electrically connection/coupling, communicatively connection/coupling, operatively connection/coupling, physically connection/coupling, and the like. The terms should be interpreted accordingly depending on context in which the terms are used, but connection/coupling forms which are not intentionally or naturally excluded should be interpreted in an open-ended manner as included in the terms.

In the present specification (including the claims), when the expression “A configured to B” is used, a physical structure of an element A may have a configuration capable of executing an operation B, and a permanent or temporary setting/configuration of the element A may be configured/set to actually execute the operation B. For example, when the element A is a general-purpose processor, the processor may have a hardware configuration capable of executing the operation B and may be configured to actually execute the operation B by setting a permanent or temporary program (instruction). When the element A is a dedicated processor, a dedicated arithmetic circuit, or the like, the circuit structure of the processor may be implemented to actually execute the operation B regardless of whether a control instruction and data are actually pertained.

In the present specification (including the claims), when the terms (for example, “comprising/including”, “having”, and the like) meaning containing or possessing are used, the terms are intended as open-ended terms including a case where an objects other than objects indicated by the purpose of the terms are contained or possessed. If the objects of these terms meaning inclusion or possession are expressions that do not specify a quantity or suggests singles (expressions with an article a or an), the expressions should be interpreted as not being limited to specific numbers.

In the present specification (including the claims), even when an expression such as “one or more” or “at least one” is used in one place and an expression not specifying a quantity or implying a singular number (an expression with an article a or an) is used in another place, the latter expression is not intended to mean “one”. In general, expressions that do not specify a quantity or suggest a single (expressions with an article a or an) should be interpreted as not necessarily being limited to a specific number.

In the present specification, when it is described that a specific effect (advantage/result) is obtained for a specific configuration according to a certain embodiment, it should be understood that the effect is also obtained for one or more other embodiments having the configuration unless otherwise stated. However, presence or absence of the effect generally depends on various factors, conditions, and/or states, and it should be understood that the effect is not necessarily obtained by the configuration. The effect is obtained only by the configuration described in an example when various factors, conditions, and/or states are satisfied. The effect is not necessarily obtained in the claimed invention defining the configuration or similar configuration.

The use of the term “maximize” or the like in the present specification (including the claims) includes determining a global maximum, determining an approximation of the global maximum, determining a local maximum, and determining an approximation of the local maximum, and should be interpreted accordingly depending on the context in which the term is used. The use of the term also includes stochastically or heuristically obtaining an approximate value of these maximum values. Similarly, the use of a term such as “minimize” includes determining a global minimum, determining an approximation of the global minimum, determining a local minimum, and determining an approximation of the local minimum, and should be interpreted accordingly depending on the context in which the term is used. The use of the term also includes stochastically or heuristically obtaining an approximate value of these minimum values. Similarly, when a term such as “optimize” is used, the term includes determining a global optimum value, determining an approximate value of the global optimum value, determining a local optimum value, and determining an approximate value of the local optimum value, and should be interpreted as appropriate according to the context in which the term is used. The use of the term also includes stochastically or heuristically obtaining an approximate value of these optimum values.

In the present specification (including the claims), when a plurality of pieces of hardware executes predetermined processes, the pieces of hardware may execute the predetermined processes in cooperation with each other, or some pieces of hardware may execute all of the predetermined processes. Some of the pieces of hardware may execute some of the predetermined processes, and the other hardware may execute the rest of the predetermined processes. In the present specification (including the claims), when an expression such as “one or more pieces of hardware execute a first process, and the one or more pieces of hardware execute a second process” is used, the hardware that executes the first process and the hardware that executes the second process may be the same or different. That is, the hardware that executes the first process and the hardware that executes the second process may be included in the one or more pieces of hardware. The hardware may include an electronic circuit or a device including an electronic circuit.

In the present specification (including the claims), when a plurality of storage devices (memories) store data, each storage device (memory) among the plurality of storage devices (memories) may store only part of the data or may store all of the data.

Although the embodiments of the present disclosure have been described in detail above, the present disclosure is not limited to the individual embodiments described above. Various additions, modifications, substitutions, partial deletions, and the like can be made without departing from the conceptual idea and gist of the present invention derived from the contents defined in the claims and equivalents thereof. For example, in all the embodiments described above, when numerical values or mathematical expressions are used for description, the numerical values or mathematical expressions are shown as examples, and the embodiments are not limited thereto. The orders of the operations in the embodiments are illustrated as examples, and the present invention is not limited thereto.

What is claimed is:

1. A device comprising:
at least one memory; and
at least one processor, wherein
the at least one processor is configured to
input a first atomic structure into a function having a parameter set and generate at least one of a second energy or a second force corresponding to the first atomic structure, and
update the parameter set based on at least one of a difference between the second energy and a first energy generated by inputting the first atomic structure into a trained model, or a difference between the second energy force and a first force generated by inputting the first atomic structure into the trained model.

2. The device according to claim 1, wherein the at least one processor is configured to generate the first atomic structure based on a second atomic structure and the parameter set.

3. The device according to claim 1, wherein the at least one processor is configured to
generate at least one of a fourth energy or a fourth force corresponding to a third atomic structure different from the first atomic structure based on the function having the updated parameter set, and the third atomic structure, and
evaluate the updated parameter set based on at least one of a difference between the fourth energy and a third energy corresponding to the third atomic structure different from the first atomic structure or a difference between the fourth force and a third force corresponding to the third atomic structure different from the first atomic structure.

4. The device according to claim 3, wherein the at least one processor is configured to perform the evaluation of the updated parameter set by validating generality of the updated parameter set.

5. The device according to claim 4, wherein the at least one processor is configured to, when the generality of the updated parameter set does not meet a given standard,
generate at least one of a sixth energy or a sixth force corresponding to a fourth atomic structure based on the function having the updated parameter set, and the fourth atomic structure, and
update the updated parameter set based on at least one of a difference between the sixth energy and a fifth energy corresponding to the fourth atomic structure different from the first atomic structure or a difference between the sixth force and a fifth force corresponding to the fourth atomic structure different from the first atomic structure.

6. The device according to claim 1, wherein the at least one processor is configured to
generate a fifth atomic structure different from the first atomic structure based on a second atomic structure and the updated parameter set,
generate at least one of an eighth energy or an eighth force corresponding to the fifth atomic structure based on the function having the updated parameter set, and the fifth atomic structure, and
update the updated parameter set based on at least one of a difference between the eighth energy and a seventh energy corresponding to the fifth atomic structure different from the first atomic structure or a difference between the eighth force and a seventh force corresponding to the fifth atomic structure different from the first atomic structure.

7. The device according to claim 1, wherein the function includes at least a neural network.

8. The device according to claim 7, wherein the parameter set is a set of weights for the neural network.

9. A device according to claim 2, wherein the second atomic structure is generated from a notation regarding a substance.

10. The device according to claim 1, wherein the function is a classical potential.

11. The device according to claim 1, the at least one processor is further configured to generate, by inputting the first atomic structure into the trained model, at least one of the first energy or the first force corresponding to the first atomic structure.

12. The device according to claim 11, wherein the trained model is a neural network potential (NNP).

13. A method comprising:
inputting, by at least one processor, the first atomic structure into a function having a parameter set and generating, by the at least one processor, at least one of a second energy or a second force corresponding to the first atomic structure, and
updating, by the at least one processor, the parameter set based on at least one of a difference between the second energy and a first energy generated by inputting the first atomic structure into a trained model, or a difference between the second energy force and a first force generated by inputting the first atomic structure into the trained model.

14. The method according to claim 13, further comprising:
generating, by the at least one processor, the first atomic structure based on a second atomic structure and the parameter set.

15. The method according to claim 13, further comprising:
calculate, by the at least one processor, a fourth energy or a fourth force corresponding to the third atomic structure different from the first atomic structure based on the function having the updated parameter set, and the third atomic structure, and
evaluate, by the at least one processor, the updated parameter set based on at least one of a difference between the fourth energy and a third energy corresponding to the third atomic structure different from the first atomic structure or a difference between the fourth force and a third force corresponding to the third atomic structure different from the first atomic structure.

16. The method according to claim 13, wherein the function includes at least a neural network.

17. The method according to claim 13, wherein the function is a classical potential.

18. The method according to claim 13, wherein the function is an optimized potentials for liquid simulations (OPLS) potential.

19. The method according to claim 14, wherein the second atomic structure is generated from a notation regarding a substance.

20. A non-transitory computer-readable storage medium for storing a program that, when executed by one or more processors of one or more computers, cause the one or more computers to:
input a first atomic structure into a function having a parameter set and generate at least one of a second energy or a second force corresponding to the first atomic structure, and
update the parameter set based on at least one of a difference between the second energy and a first energy generated by inputting the first atomic structure into a trained model, or a difference between the second energy force and a first force generated by inputting the first atomic structure into the trained model.
